(19) United States
(12) Patent Application Publication
(10) Pub. No.: US 2004/0215328 A1
(43) Pub. Date: Oct. 28, 2004

(54) Bifurcated stent with concentric body portions

(76) Inventor: Ronan Thornton, Galway (IE)

Correspondence Address: Cardinal Law Group, Suite 2000, 1603 Orrington Avenue, Evanston, IL, 60201, US

(21) Appl. No.: 10/423,840

(22) Filed: Apr. 25, 2003

(51) Int. Cl. A61F 2/06

(52) U.S. Cl. 623/1.16; 623/1.35

(57) ABSTRACT

The present invention provides a bifurcated stent comprising first and second stent frameworks. The stent frameworks may be cut from tubular stent frameworks using a laser. Each stent framework includes a leg portion and a body portion. The body portions are formed with segments and gaps and are concentrically aligned such that there is minimal overlap of the segments. Each leg portion may be crimped independently of its corresponding body portion, thus achieving a diameter less than that of its corresponding body portion. The leg portions may be capable of deflecting one from the other when the body portions are concentrically aligned.

### TECHNICAL FIELD

This invention relates generally to biomedical stents. More specifically, the invention relates to a bifurcated stent comprising two stent frameworks, each framework including a leg portion and a body portion. The body portions are formed with segments and gaps that are concentrically aligned to provide minimal overlap of segments.

### BACKGROUND OF THE INVENTION

Stents are generally cylindrical-shaped devices that are radially expandable to hold open a segment of a vessel or other anatomical lumen after implantation into the lumen. Various types of stents are in use, including expandable and self-expanding stents. Expandable stents generally are conveyed to the area to be treated on balloon catheters or other expandable devices. For insertion, the stent is positioned in a compressed configuration along the delivery device, for example crimped onto a balloon that is folded or otherwise wrapped about a guide wire that is part of the delivery device. After the stent is positioned across the lesion, it is expanded by the delivery device, causing the diameter of the stent to expand. For a self-expanding stent, commonly a sheath is retracted, allowing expansion of the stent.

Stents are used in conjunction with balloon catheters in a variety of medical therapeutic applications, including intravascular angioplasty. For example, a balloon catheter device is inflated during percutaneous transluminal coronary angioplasty (PTCA) to dilate a stenotic blood vessel. The stenosis may be the result of a lesion such as a plaque or thrombus. When inflated, the pressurized balloon exerts a compressive force on the lesion, thereby increasing the inner diameter of the affected vessel. The increased interior vessel diameter facilitates improved blood flow.

Soon after the procedure, however, a significant proportion of treated vessels restenose. To prevent restenosis, a stent, constructed of a metal or polymer, is implanted within the vessel to maintain lumen size. The stent acts as a scaffold to support the lumen in an open position. Configurations of stents include a cylindrical tube defined by a solid wall, a mesh, interconnected stents, or like segments. Exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz, and U.S. Pat. No. 5,421,955 to Lau.

Difficulties arise when the area requiring treatment is located near a bifurcation, the point at which a single vessel branches into two vessels. To effectively treat a vascular condition at a bifurcation, the stent must cover the entire affected area without obstructing blood flow in either adjoining vessel. This can be quite difficult to achieve.

Various conventional stenting techniques have been disclosed for treating bifurcations. One conventional bifurcation stenting technique includes first stenting the side-branch vessel and then the main vessel. Angle variations or limited visualization at the ostium (area at the opening) of the side-branch vessel may prevent accurate placement of the side-branch stent, resulting in the stent providing suboptimal coverage of the ostium or in the stent protruding into the main vessel and interfering with blood flow. The stent may, additionally, block access to portions of the adjoining vessel that require further intervention.

Another conventional technique involves first stenting the main vessel and then advancing a second stent through the wall of the main vessel stent and into the side-branch vessel, where the second stent is deployed. Disadvantages of this method include a risk of compressing the ostium of the side branch vessel when the main vessel stent is deployed, making insertion of a second stent difficult, if not impossible. Even when the side-branch vessel remains open, accurate positioning of a second stent through the wall of the first stent and into the side branch presents significant challenges and may result in undesirable overlapping of the stents.

Where the bifurcation forms a Y-shape, with the main vessel branching into two smaller vessels, conventional techniques have included placing three stents, one within the main vessel, and one within each of the smaller vessels. The problems discussed above may be present with this technique, as well.

Therefore it would be desirable to have a bifurcated stent and a system for treating a vascular condition that overcomes the aforementioned and other disadvantages.

### SUMMARY OF THE INVENTION

One aspect of the present invention is a bifurcated stent comprising first and second stent frameworks. Each stent framework includes a leg portion and a body portion. The body portions are formed with segments and gaps and are concentrically aligned such that there is minimal overlap of the segments.

Another aspect of the present invention is a system for treating a vascular condition, comprising a catheter and first and second stent frameworks operably coupled to the catheter. Each stent framework includes a leg portion and a body portion. The body portions are formed with segments and gaps and are concentrically aligned such that there is minimal overlap of the segments.

A further aspect of the present invention is a method of manufacturing a bifurcated stent. A first stent framework is provided that includes a first leg portion and a first body portion. A second stent framework is provided that includes a second leg portion and a second body portion. Each leg portion is crimped to a diameter less than that of the corresponding body portion. The first and second body portions are concentrically aligned.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration of one embodiment of a first stent framework, in accordance with the present invention;

FIG. 2 is an illustration of one embodiment of a second stent framework, in accordance with the present invention;

FIG. 3 is an illustration of the stent framework of FIG. 1, showing the first stent framework after the first leg portion has been crimped to a diameter less than that of the first body portion;

FIG. 4 is an illustration of the stent framework of FIG. 2, showing the second stent framework after the second leg portion has been crimped to a diameter less than that of the second body portion;

FIG. 5 is an illustration of the first and second stent frameworks of FIG. 3 and FIG. 4 in position to be concentrically aligned;

FIG. 6 is an illustration of one embodiment of a bifurcated stent formed using the stent frameworks shown in FIG. 5, in accordance with the present invention;

FIG. 7 is an illustration of a system for treating a vascular condition, in accordance with the present invention; and

FIG. 8 is a flow diagram of one embodiment of a method of manufacturing a bifurcated stent, in accordance with the present invention.

### DETAILED DESCRIPTION OF THE PRESENTLY PREFERRED EMBODIMENTS

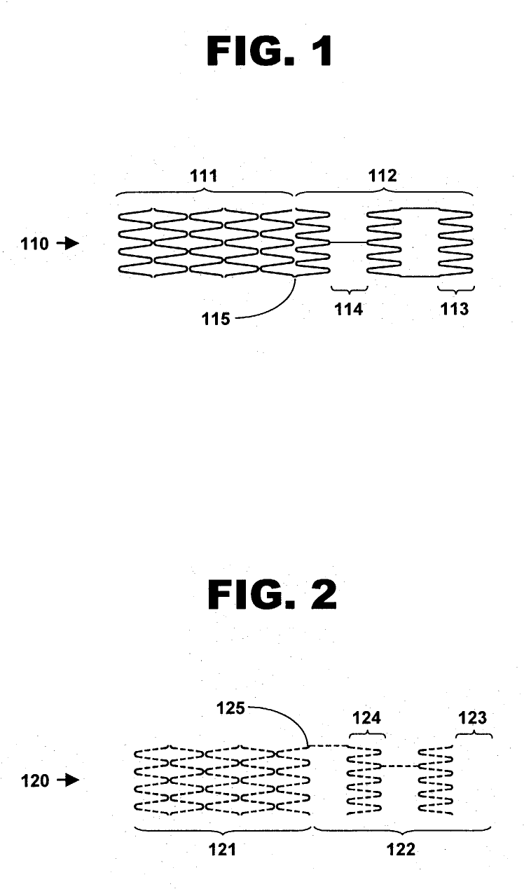

One aspect of the present invention is a bifurcated stent. One embodiment of the stent, in accordance with the present invention, is illustrated in FIGS. 1 through 6. Like elements share like reference numbers in the figures.

The bifurcated stent of the present invention comprises a first stent framework 110 and a second stent framework 120. As shown in FIG. 1, first stent framework 110 includes a first leg portion 111 and a first body portion 112. The first body portion is formed with segments 113 and gaps 114. In this embodiment, first leg portion 111 is attached to first body portion 112 at a single point 115, which allows leg portion 111 to be crimped independently of body portion 112.

As seen in FIG. 2, second stent framework 120 includes a second leg portion 121 and a second body portion 122. The second body portion is formed with segments 124 and gaps 123. Second leg portion 121 is attached to second body portion 122 at a single point 125 to allow leg portion 121 to be crimped independently of body portion 122.

The stent frameworks may be made of a wide variety of medical implantable materials, such as stainless steel, nitinol, tantalum, ceramic, nickel, titanium, aluminum, polymeric materials, tantalum, MP35N, stainless steel, titanium ASTM F63-83 Grade 1, niobium, high carat gold K 19-22, or combinations of the above. The stent frameworks may be formed by cutting the patterns seen in FIG. 1 and FIG. 2 into the walls of tubular stent frameworks using a laser.

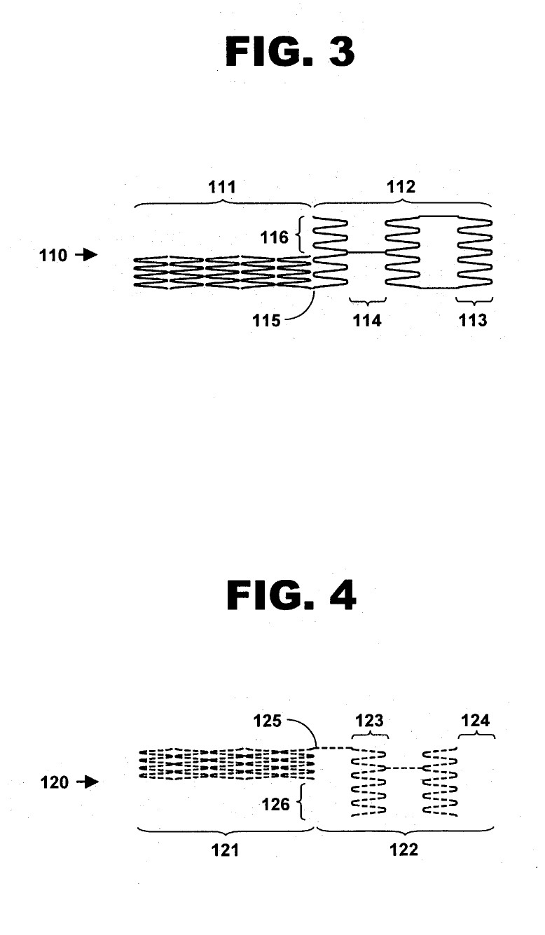

FIG. 3 shows first stent framework 110 with first leg portion 111 crimped to a diameter less than that of its corresponding body portion 112. In FIG. 4, second leg portion 121 of second stent framework 120 has also been crimped to a diameter less than that of its corresponding body portion 122. In both FIG. 3 and FIG. 4, the leg portions are shown crimped to a diameter slightly less than one-half that of the corresponding body portion. As can be seen, crimping of the leg portions leaves an opening 116 in first body portion 112 and an opening 126 in second body portion 122.

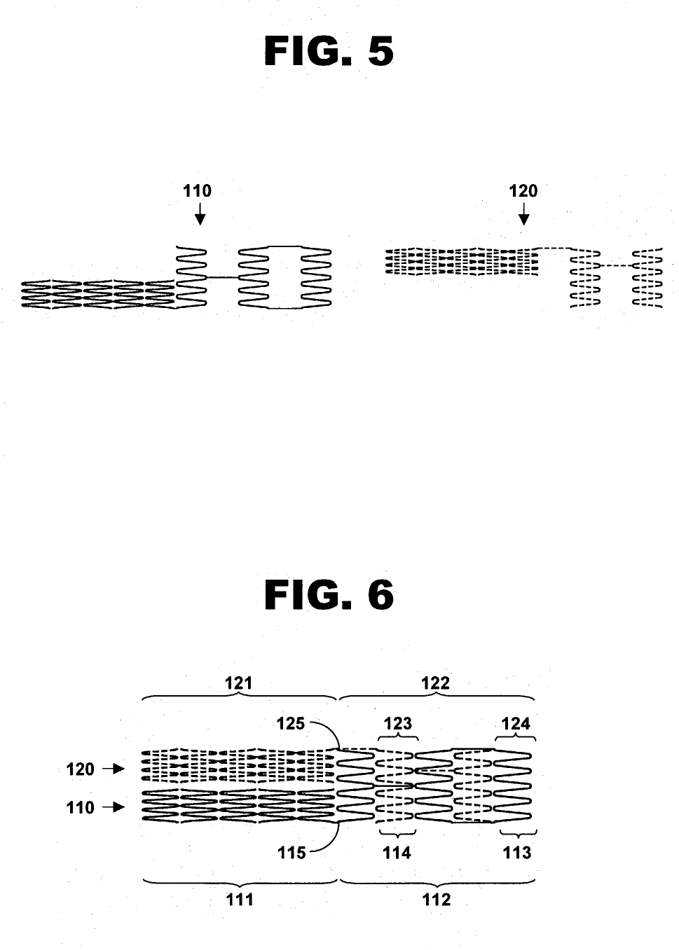

FIG. 5 illustrates first stent framework 110 and second stent framework 120 in position to be concentrically aligned. In FIG. 6, first stent framework 110 and second stent framework 120 have been assembled to form a bifurcated stent. The concentric alignment of first body portion 112 with second body portion 122 has been achieved by inserting second stent framework 120 into first stent framework 110, with second leg portion 121 passing through opening 116. Thus, second body portion 122 is within and concentrically aligned with first body portion 112. At least a distal end of body portion 122 may be crimped to facilitate inserting body portion 122 into body portion 112.

As can be seen, the segments 113 and gaps 114 of first body portion 112 and the segments 123 and gaps 124 of second body portion 122 align such that there is minimal overlap of the segments. Linkage points 115 and 125 are positioned to allow leg portions 111 and 121 to deflect one from the other.

A bifurcated stent in accordance with the present invention may include a therapeutic agent (not shown) disposed on at least a portion of the stent. The agent may be, for example, an antineoplastic agent, an antiproliferative agent, an antibiotic, an anti-inflammatory agent, combinations thereof, and the like.

The stent may further include a graft member (also not shown) such as a thin sleeve of polyester, expanded polytetrafluoroethylene (PTFE), or other appropriate material. The graft member may be positioned between the two body portions to act as a cushion between them, or it may be used to provide therapeutic benefits such as improved scaffolding or local delivery of a therapeutic agent in the vicinity of the bifurcation.

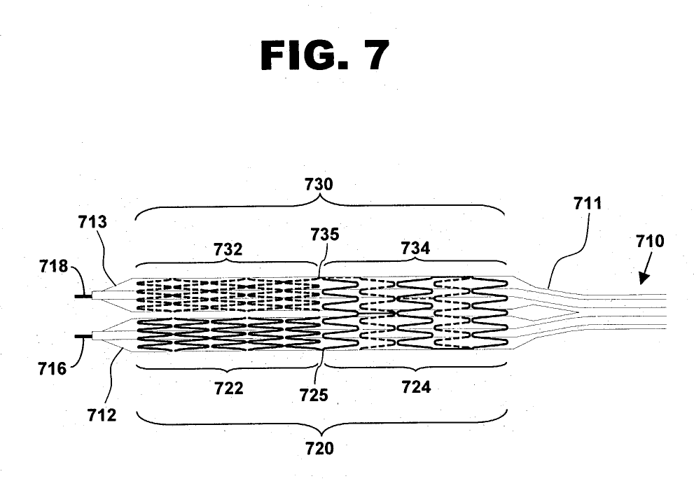

Another aspect of the present invention is a system for treating a vascular condition. One embodiment of the system, in accordance with the present invention, is illustrated in FIG. 7. The system comprises a catheter 710, a first stent framework 720, and a second stent framework 730.

Catheter 710 may be any catheter known in the art that is appropriate for delivering a stent to a treatment site within a vessel, for example a percutaneous transluminal coronary angioplasty (PTCA) balloon catheter.

First stent framework 720 and second stent framework 730 may be made of a wide variety of medical implantable materials, such as stainless steel, nitinol, tantalum, ceramic, nickel, titanium, aluminum, polymeric materials, tantalum, MP35N, stainless steel, titanium ASTM F63-83 Grade 1, niobium, high carat gold K 19-22, or combinations of the above. The stent frameworks may be formed by cutting patterns into tubular wall stent frameworks using a laser.

First stent framework 720 includes a first leg portion 722 and a first body portion 724. Second stent framework 730 includes a second leg portion 732 and a second body portion 734. Each leg portion is crimped to a diameter less than that of its corresponding body portion. The first body portion is linked to the first leg portion at 725 and the second body portion is linked to the second leg portion at 735, these points positioned to allow the leg portions to be crimped independently of the body portions. The linkage points are additionally positioned to allow the leg portions to deflect one from the other when the body portions are aligned, facilitating placement of each leg portion within a branch of a bifurcated vessel.

Body portions 724 and 734 are formed with segments and gaps. The two body portions are concentrically aligned such that there is minimal overlap of the segments. The concentric alignment may be achieved by inserting second stent framework 730 into first stent framework 720, with second leg portion 732 passing through an opening formed in first body portion 724 by the crimping of first leg portion 722. Thus, second body portion 734 is positioned within and concentrically aligned with first body portion 724. At least a distal end of second body portion 734 may be crimped to facilitate inserting body portion 734 into body portion 724.

In the embodiment shown in FIG. 7, catheter 710 includes a bifurcated balloon 711, which comprises a first balloon portion 712 and a second balloon portion 713. Bifurcated balloon 711 may be made of a suitable material such as polyethylene, polyethylene terephthalate (PET), nylon®, pebax®, or the like. The dimensions of balloon 711 may be selected based on the dimensions of the stent being delivered.

First leg portion 722 is operably coupled to first balloon portion 712. Second leg portion 732 is operably coupled to second balloon portion 713. The concentrically aligned first and second body portions 724 and 734 are operably coupled to both balloon portions, with the two balloon portions adjacent each other within the concentric body portions.

The system further includes a first guide wire 716 and a second guide wire 718. First guide wire 716 runs through a lumen within first balloon portion 712, while second guide wire 718 runs through a lumen within second balloon portion 713. The two guide wires aid in positioning leg portions 722 and 732 within the branches of a bifurcated vessel.

A system in accordance with the present invention may include a therapeutic agent (not shown) disposed on at least a portion of the system. The agent may be, for example, an antineoplastic agent, an antiproliferative agent, an antibiotic, an anti-inflammatory agent, or combinations thereof, and the like.

The system may further include a graft member (also not shown) such as a thin sleeve of polyester, expanded polytetrafluoroethylene (PTFE), or other appropriate material. The graft member may be positioned between the two body portions to act as a cushion between them, or it may be used to provide therapeutic benefits such as improved scaffolding or local delivery of a therapeutic agent in the vicinity of the bifurcation.

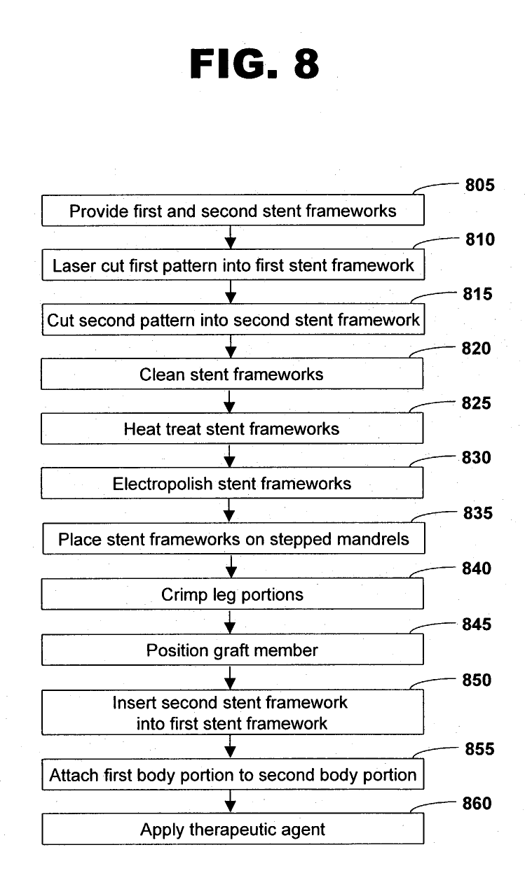

A further aspect of the present invention is a method of manufacturing a bifurcated stent. FIG. 8 shows a flow diagram of one embodiment of a method of manufacturing a bifurcated stent, in accordance with the present invention.

First and second stent frameworks are provided, each framework including a leg portion and a body portion (Block 805). Providing such a stent framework may include, for example, laser cutting a pattern through the wall of a tubular stent framework. A first pattern may be cut into the first stent framework (Block 810), while a second pattern may be cut into the second stent framework (Block 815). Both stent frameworks may be fully processed as for any laser cut stent. Thus, the stent frameworks maybe cleaned (Block 820), heat treated (Block 815), and electropolished (Block 830) following laser cutting.

The pattern cut into the body portion of the first stent framework forms segments and gaps in the framework. The pattern cut into the body portion of the second stent framework also forms segments and gaps in the framework. These segments and gaps are positioned such that the segments of one body portion fill the gaps of the other body portion, thus providing minimal overlap of the segments when the two body portions are concentrically aligned. One skilled in the art will recognize that a wide variety of patterns may be devised to accomplish this and to provide other capabilities desired for the stent.

After laser cutting, each stent framework may be placed on a stepped mandrel for crimping (Block 835). An appropriate stepped mandrel would comprise two cylindrical portions, the first sized to fit snugly inside the stent framework, and the second having a diameter somewhat less than half that of the first mandrel portion. Each leg portion is then crimped down onto the mandrel portion having the smaller diameter (Block 840). This results in each leg portion having a diameter less than that of its corresponding body portion.

The patterns cut into the leg portions are designed not only to permit each leg portion to be crimped independently of its corresponding body portion, but also to enable the leg portions to deflect one from the other when the body portions are concentrically aligned. This ability to deflect facilitates placement of the leg portions within the branches of a bifurcated vessel. The leg and body portions may be linked at a single point or adjacent points on the stent framework, with the point(s) oriented appropriately on the mandrel during the crimping step.

A graft member may be positioned adjacent the second body portion prior to alignment of the two body portions (Block 845). The graft member may act as a cushion between the two body portions or may provide therapeutic benefits such as improved scaffolding and delivery of a therapeutic agent.

Concentric alignment of the body portions may be achieved by, for example, inserting the second stent framework into the first stent framework (Block 850), with the second leg portion passing through the opening formed in the first body portion when the first leg portion was crimped. The second body portion is thus positioned within and concentrically aligned with the first body portion, the segments of each body portion filling the gaps of the other body portion such that there is minimal overlap of the segments. At least a distal end of the second body portion may be crimped to facilitate insertion.

The body portion of the first stent framework may be attached to the body portion of the second stent framework using a method such as spot welding (Block 855). Such attachment may secure the stent frameworks one to the other, reducing or eliminating the risk of fretting between the two frameworks.

A therapeutic agent may be applied to provide local delivery of a therapeutic agent in the vicinity of the bifurcation (Block 860). The therapeutic agent may be applied to all or a portion of one or both stent frameworks. It may also be applied to all or a portion of the graft member. An application method such as dipping, spraying, pad printing, inkjet printing, rolling, painting, micro-spraying, wiping, electrostatic deposition, vapor deposition, epitaxial growth, and combinations thereof may be used. One skilled in the art will recognize that a therapeutic agent may be applied before, during, or after assembly of the stent.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A bifurcated stent comprising:
a first stent framework including a first leg portion and a first body portion; and
a second stent framework including a second leg portion and a second body portion, wherein the first and second body portions are formed with segments and gaps and are concentrically aligned such that there is minimal overlap of the segments.

2. The stent of claim 1 wherein each of the first stent framework and the second stent framework is formed by laser cutting a pattern into a tubular wall stent framework.

3. The stent of claim 1 wherein each leg portion is crimped to a diameter less than that of the corresponding body portion.

4. The stent of claim 1 wherein the first body portion is linked to the first leg portion and the second body portion is linked to the second leg portion at points positioned to allow the leg portions to be crimped independently of the body portions.

5. The stent of claim 4 wherein the points of linkage are positioned to allow the leg portions to deflect one from the other when the first and second body portions are concentrically aligned.

6. The stent of claim 1 wherein at least a distal end of one of the first body portion and the second body portion is crimped to facilitate concentric alignment of the body portions.

7. The stent of claim 1 further comprising:
a graft member positioned adjacent at least a portion of one of the first stent framework and the second stent framework.

8. The stent of claim 1 further comprising:
a therapeutic agent disposed on at least a portion of the stent.

9. A system for treating a vascular condition, comprising:
a catheter;
a first stent framework operably coupled to the catheter, the first stent framework including a first leg portion and a first body portion; and
a second stent framework operably coupled to the catheter, the second stent framework including a second leg portion and a second body portion, wherein the first and second body portions are formed with segments and gaps and are concentrically aligned such that there is minimal overlap of the segments.

10. The system of claim 9 wherein each of the first stent framework and the second stent framework is formed by laser cutting a pattern into a tubular wall stent framework.

11. The system of claim 9 wherein each leg portion is crimped to a diameter less than that of the corresponding body portion.

12. The system of claim 9 wherein the first body portion is linked to the first leg portion and the second body portion is linked to the second leg portion, each at a point positioned to allow the leg portion to be crimped independently of the body portion.

13. The system of claim 12 wherein the points of linkage are positioned to allow the first and second leg portions to deflect one from the other when the first and second body portions are concentrically aligned.

14. The system of claim 9 wherein at least a distal end of one of the first body portion and the second body portion is crimped to facilitate concentric alignment of the body portions.

15. The system of claim 9 wherein the catheter includes a bifurcated balloon having a first balloon portion and a second balloon portion.

16. The system of claim 15 wherein the first leg portion is operably coupled to the first balloon portion, the second leg portion is operably coupled to the second balloon portion, and the concentrically aligned body portions are operably coupled to the first and second balloon portions together.

17. The system of claim 15 further comprising:
a first guide wire; and
a second guide wire.

18. The system of claim 9 further comprising:
a graft member positioned adjacent at least a portion of one of the first stent framework and the second stent framework.

19. The system of claim 9 further comprising:
a therapeutic agent disposed on at least a portion of the system.

20. A method of manufacturing a bifurcated stent, comprising:
providing a first stent framework including a first leg portion and a first body portion;
providing a second stent framework including a second leg portion and a second body portion;
crimping each leg portion to a diameter less than that of the corresponding body portion; and
concentrically aligning the first and second body portions.

21. The method of claim 20 wherein providing a first stent framework includes laser cutting a first pattern into a tubular wall stent framework.

22. The method of claim 21 wherein providing a first stent framework further includes performing steps selected from a group consisting of cleaning the stent framework, heating the stent framework, electropolishing the stent framework, and combinations thereof.

23. The method of claim 20 wherein providing a second stent framework includes laser cutting a second pattern into a tubular wall stent framework.

24. The method of claim 23 wherein providing a second stent framework further includes performing steps selected from a group consisting of cleaning the stent framework, heating the stent framework, electropolishing the stent framework, and combinations thereof.

25. The method of claim 20 wherein providing a first stent framework includes forming the first body portion with segments and gaps.

26. The method of claim 25 wherein providing a second stent framework includes forming the second body portion with segments and gaps such that there is minimal or no overlap of segments when the second body portion is concentrically aligned with the first body portion.

27. The method of claim 20 further comprising:
crimping at least a distal end of one of the first body portion and the second body portion to facilitate concentric alignment of the body portions.

28. The method of claim 20 further comprising:
positioning a graft member adjacent at least a portion of one of the first stent framework and the second stent framework.

29. The method of claim 20 further comprising:
applying a therapeutic agent to at least a portion of the stent.

30. The method of claim 20 further comprising:
attaching the body portion of the first stent framework to the body portion of the second stent framework.
